(19) United States
(12) Patent Application Publication
(10) Pub. No.: US 2021/0267459 A1
(43) Pub. Date: Sep. 2, 2021

(54) DETERMINATION OF THE DEGREE OF REDUCTIVE DAMAGE OF HAIR WITH NIR SPECTROSCOPY

(71) Applicant: HENKEL AG & CO. KGAA, Duesseldorf (DE)

(72) Inventors: Hans Georg KNUEBEL, Duesseldorf (DE); Lucile BONNIN, Berlin (DE)

(73) Assignee: Henkel AG & Co. KGaA, Duesseldorf (DE)

(21) Appl. No.: 17/054,003

(22) PCT Filed: Apr. 26, 2019
(86) PCT No.: PCT/EP2019/060734

§ 371 Date: Nov. 9, 2020

(30) Foreign Application Priority Data
May 16, 2018 (DE) 10 2018 207 560.3

(51) Int. Cl. A61B 5/00; G01N 21/359; G01N 21/3563

(52) U.S. Cl. CPC A61B 5/0075; G01N 21/3563; G01N 21/359; A61B 5/448

(57) ABSTRACT

An arrangement to determine a reductive damage of hair is provided. The arrangement contains a detection unit for detecting hair features and an evaluation unit for evaluating the detected hair features and for determining the reductive damage of hair based on the detected hair features. The detection unit contains a near infrared sensor, and is configured to irradiate a hair sample with electromagnetic waves in the near infrared range at a wavelength from about 800 to about 2500 nm and to detect the light emitted by the hair sample. The detection unit is configured to generate a spectrum of the emitted light and provide it to the evaluation unit and the evaluation unit is configured to determine a reductive damage based on the spectrum of the emitted light in a wavelength range from about 1970 to about 1980 nm.

### CROSS-REFERENCE TO RELATED APPLICATION

This application is a U.S. National-Stage entry under 35 U.S.C. § 371 based on International Application No. PCT/EP2019/060734, filed Apr. 26, 2019, which was published under PCT Article 21(2) and which claims priority to German Application No. 10 2018 207 560.3, filed May 16, 2018, which are all hereby incorporated in their entirety by reference.

### TECHNICAL FIELD

The present disclosure relates to an arrangement for determining a reductive damage of hair, a method for determining the reductive damage of hair, and a computer program product which is adapted to carry out the steps of such a method when executed on a corresponding arrangement.

### BACKGROUND

When hair is treated with cosmetic products, the effect of the product, e.g. the intensity of a coloring, can depend very much on the damage to the hair.

Hair can be damaged by natural or artificial processes. Natural processes can include, for example, a combined (e.g. simultaneous) action of UV light and oxygen (O₂) on the hair. The artificially induced processes may include, for example, the application of hair dyes (also known as coloring), bleaching, and/or the creation of a permanent wave.

In addition to desired cosmetic effects, such as a lightening of the hair, this can also cause severe damage to the hair, for example when using oxidizing agents.

The resulting type of damage is called oxidative damage and is caused by the application of coloring, bleaching, permanent waves and by environmental influences (UV+O₂). This damage is caused by the oxidation of the amino acids cystine and cysteine, which are common in hair, to cysteic acid.

Cystine can form intermolecular disulfide bridges (also known as S—S bridges) in the hair, so cystine is extremely important for the mechanical stability of the hair.

The oxidation of these bridges to cysteic acid can destroy the mechanical stability of the hair and even lead to complete hair breakage if used several times. However, macroscopically perceptible, e.g. tactile, properties of the hair, such as surface roughness, can be negatively influenced. The results of cosmetic treatments, especially damaging procedures, can also be massively changed at an early stage of damage compared to the results with undamaged hair.

Besides this oxidative damage, however, reductive damage to the hair is also possible. This occurs in cosmetic procedures that use reducing agents. These are, for example, perms or straighteners containing reducing agents such as thioglycolic acid or sulfite. These ingredients serve to open the disulfide bridges of cysteine to reshape the hair. The following sulfur species are formed: R—S—H (thiols), R—S—SO₃—, (colored salts, after sulfite treatment), R—S—S—CH₂COO⁻ (disulfides with thiglycolate units, after thioglycolate treatment).

In an academic and industrial environment, a researcher or developer may have a variety of physical and chemical-analytical methods at his disposal to determine the degree of damage to hair. Conventional chromatographic methods are used here, such as high-performance liquid chromatography (HPLC) after a complex acidic hydrolytic digestion of the hair sample.

Undamaged hair can typically have a cysteic acid content ranging from about 0.5% to about 1% (by weight). In the presence of damage, for example because of multiple ultra-blonding and/or other damage mechanisms, the cysteic acid content can rise to over about 15% (by weight).

However, all these chromatographic methods are complicated and expensive in terms of equipment, so they are not available to the end user.

Harmful cosmetic treatments, such as hair coloring, heat treatments, permanent waves, or oxidative procedures such as bleaching, and many others, are typically carried out in the private sector or in the field of commercial services to the end consumer. Although carrying out another damaging procedure on pre-damaged hair can lead to catastrophic results, up to and including complete hair breakage, there has so far been no way of determining the degree of pre-damage to the hair, for example quantitatively.

Furthermore, there are many different hair treatment products on the market which are designed to improve different hair properties or parameters, such as shine. In many cases, however, the user of such products is not aware of the damage to the hair. This can lead to the user resorting to products that are less suitable in his case and being dissatisfied with their effectiveness after use.

Therefore, an investigation of damage to the hair can be of great importance. It can also be advantageous to offer a product tailored to individual needs.

### BRIEF SUMMARY

Arrangements and methods for determining a reductive damage of hair are provided herein. In an exemplary embodiment, an arrangement for determining a reductive damage of hair includes a detection unit for detecting hair characteristics thereby generating detected hair characteristics. The arrangement further includes, but is not limited to, an evaluation unit for evaluating the detected hair characteristics and for determining the reductive damage of hair based on the detected hair characteristics. The detection unit includes a near infrared sensor, NIRS. The detection unit is configured to irradiate a hair sample with electromagnetic waves in the near infrared region at a wavelength of from about 800 to about 2500 nm and to detect light emitted from the hair sample. The detection unit is configured to generate a spectrum of the emitted light and to provide the spectrum to the evaluation unit. The evaluation unit is configured to determine a reductive damage based on the spectrum of the emitted light in a wavelength range of from about 1970 to about 1980 nm.

### BRIEF DESCRIPTION OF THE DRAWINGS

The present disclosure will hereinafter be described in conjunction with the following drawing figures, wherein like numerals denote like elements, and:

FIG. 1 is a schematic representation of an arrangement to determine a reductive damage of hair according to an execution example;

FIG. 2 is a schematic representation of a data carrier according to a further design example;

FIG. 3 is a schematic representation of the steps of a procedure according to a further execution example; and

FIG. 4 is a schematic representation of an acquisition unit for an arrangement according to a further design example.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

It can be regarded as the task of the present disclosure to simplify the determination of a reductive damage of hair, especially regarding the expenditure of apparatus and time.

This task is solved with the characteristics of independent claims. Further developments of the present disclosure result from the dependent claims and from the following description.

The present disclosure is based, inter alia, on the following findings: It was found that the sulfur species formed during a reductive treatment of hair can be exemplified by vibrational spectroscopy.

However, since an oxidative fixation step often still occurs after reductive treatments, an increase in cysteic acid units in the hair proteins can be observed in addition to the functional groups described above. The analysis of such a complex chemistry can be done with vibrational spectroscopic methods using chemometric models. The model can be calibrated by external quantification, e.g. by HPLC, of the species described above, or by assigning a hair condition to the calibration samples based on the hair treatment performed (e.g. 1× light perm, 2× strong perm, etc.) The chemometric model then does not quantitatively determine a chemical species but assigns an ordinal or categorical data value to the unknown hair samples based on the spectral data.

According to one aspect, an order to determine a reductive damage of hair is given. The arrangement has a detection unit for detecting hair characteristics and an evaluation unit for evaluating the detected hair characteristics and determining the reductive damage of hair based on the detected hair characteristics. The detection unit contains a near infrared sensor (NIRS) and is designed to irradiate a hair sample with electromagnetic waves in the near infrared range at a wavelength between about 800 and about 2500 nm and to detect the light emitted by the hair sample.

The detection unit is further designed to generate a spectrum of the light emitted and make it available to the evaluation unit. The evaluation unit is designed to determine a reductive damage based on the spectrum of the emitted light in a wavelength range between about 1970 and about 1980 nm.

It has been recognized that thiols in hair, when exposed to light in the near-infrared range, have a harmonic in the range from about 1970 to about 1980 nm. From the spectrum of the emitted light, this wavelength range can be used to determine the extent of the reductive damage. The detection unit detects at least this wavelength range in the spectrum of the light emitted by the hair sample.

Spectra can basically be measured in transmission or reflection. Reflection detects the electromagnetic waves reflected from a sample and transmission detects the electromagnetic waves passing/transmitting the sample. For the purposes of this description, reflected or passing/transmitting electromagnetic waves are referred to as emitted electromagnetic waves or emitted light.

Reductive damage to hair is defined as damage to hair caused using reducing agents or treatment products containing reducing agents.

The evaluation unit can be a processor or computer that implements and executes signal processing algorithms to evaluate and process the spectrum of the light emitted.

A hair sample can be a single hair, but preferably contains several individual hairs to increase the intensity of the emitted light (or the amount of emitted light relative to the emitted light) compared to a single hair. For example, hair can be examined non-destructively by pointing the detection unit at a person's scalp hair. In other words, the hair can be examined without having to cut it off.

A spectrum of light is understood to be the intensity of light over frequency or wavelength. The spectrum can be a continuous indication of the intensity of light in a frequency or wavelength range from a lower value to an upper value. This continuous indication describes the intensity value of the light over the wavelength or frequency and has a course characteristic of the chemical composition or damage of the hair sample.

In particular, the damage or harm can be a value, for example a scalar value, which indicates the extent of the damage or the category of damage (e.g. non-existent, low, medium, strong, extraordinarily strong).

According to one design, the detection unit is designed to emit the light emitted in the direction of the hair sample to be examined over the entire specified wavelength range between about 800 and about 2500 nm.

The hair sample is therefore illuminated in a broadband way so that the emitted light, the answer, can also be recorded in a broadband way. It is conceivable that not only the spectrum alone in a narrow wavelength range is used, but also the spectral pattern starting from the above-mentioned narrow range of oscillations of thiol.

According to another version, the evaluation unit is designed to set an intensity of the emitted light in the wavelength range between about 1970 and about 1980 nm in relation to the wavelength ranges below and above from the wavelength range of the emitted light and to determine the reductive damage based on the pattern of the spectrum of the emitted light.

Thus, not only the absolute value of the intensity of the emitted light in the wavelength range between about 1970 and about 1980 nm is used, but also its relation to the wavelength ranges below and above. This ratio can indicate whether the proportion of thiols in the hair sample exceeds or falls below a threshold value, so that from the ratio it can be concluded about the damage of the hair sample.

For example, the spectral course (the intensity or pattern of the spectrum) between about 1970 and about 1980 nm can be related to the spectral course between about 1900 and about 1969 nm on the one hand and about 1981 and about 2050 nm on the other. The lower limit of the lower wavelength range can also be 1800, 1700, 1600, 1500 nm and further in steps of about 100 nm up to about 800 nm. The upper limit of the upper wavelength range can be about 2100, about 2200, about 2300, about 2400 or about 2500 nm.

By putting this in relation it can be determined whether the spectrum in from about 1970 to about 1980 nm has a relative maximum, a relative minimum, or no significant deviation from the intensity in the lower or higher wavelength ranges. We can speak of a relative maximum or minimum if the intensity of the emitted light in the wavelength range between from about 1970 and about 1980 nm is at least about 5% higher than the intensity in the considered areas below and above this wavelength range. The percentage deviation given refers to the intensity in the wavelength range under investigation between about 1970 and about 1980 nm. However, a deviation can also be considered significant if it deviates by at least about 10%, about 15%, about 20%, about 25% or more. It can be said that the higher the deviation, the greater the reductive damage of the hair sample.

According to another version, the evaluation unit is designed to match the spectrum of light emitted by the hair sample with known and available spectra of hair samples and to assign a damage category to the hair sample based on this match.

The known and available spectra can be part of a calibration model. For example, the calibration model can be created based on a plurality of calibration hair samples, whereby a calibration spectrum is recorded for each individual calibration hair sample (as also described above for generating the spectrum of the hair sample to be examined) and the calibration hair sample is examined for damage using other independent analytical methods, and the damage thus determined is assigned to the calibration hair sample.

By comparing the spectra of the calibration hair samples with the spectrum of the hair sample to be tested, the damage for the latter can be determined. This adjustment can consider the shape of the spectrum and/or its intensity. The consideration of the shape can also provide a hit during the comparison if the absolute values of the chemical composition of the examined hair sample and the calibration hair samples do not match.

The calibration model can be stored in a local memory of the equipment or in an external data storage unit. The detection unit records at least part of a spectrum of NIR light that has interacted with the hair sample and compares this spectrum with a plurality of calibration hair samples. For this adjustment, for example, a processor of the evaluation unit is used and at least a part of the spectrum of the examined hair sample is compared with the spectroscopic calibration model to determine the reductive damage of the hair.

For the purposes of this description, near infrared (NIR) is defined as electromagnetic waves with a wavelength between about 780 nm and about 3000 nm (both inclusive). As described elsewhere in this document, certain smaller wavelength ranges are selected from this wavelength range to create a spectrum of a hair sample and to conclude from this spectrum on the reductive damage of the hair sample.

According to another version, the near infrared sensor is a spectrometer, whereby the spectrometer is designed to generate a signal pattern based on the detected light, which is characteristic for the hair properties.

This means that it is not necessary to determine absolute values for hair properties. Rather, it may be sufficient to use the signal patterns obtained from the spectrometer to determine a product recommendation and/or application advice for the treatment and/or care of the hair. The signal pattern can be part of any calibration sample of the calibration model. Independently of this, it can be determined which products and/or application instructions are useful for hair samples with a specific signal pattern. The products and/or application instructions thus determined can then be assigned the corresponding signal pattern to express that these products and/or application instructions are suitable for hair with this signal pattern and to achieve a specific effect. This means that it is easier to find a suitable product recommendation because only the signal patterns detected in the hair sample examined need to be compared with signal patterns associated with the products and/or application and treatment instructions.

According to a further design form, the arrangement further comprises a housing and an energy storage unit, wherein the evaluation unit is accommodated in the housing and the detection unit is coupled to the housing, and wherein the energy storage unit is arranged in the housing in order to supply the evaluation unit with energy and in order to enable self-sufficient operation of the evaluation unit at least temporarily without connection to an external energy source.

This means that the evaluation unit can be operated autonomously and without external energy supply as intended. The energy store is preferably a rechargeable energy store. In one design example, the user interface is also located in the housing and can be supplied with power from the energy source, so that it can also be operated autonomously. In another design example, the energy storage unit can also supply the registration unit with energy.

The housing and the evaluation unit can be part of a user's personal device. The personal device can be a portable computer in the form of a smartphone, tablet, or other computer (these units can be commonly referred to as a computing unit or portable computing device). The detection unit can be connected to or integrated into the calculation unit and can be used to determine the degree of reductive damage to hair as described herein. In a computer program product (e.g. software or application for the personal/portable device) the recorded characteristics of the hair are then displayed in the form of values, arbitrary units, or output acoustically. The parameters can then be used to (a) derive product recommendations for individually suitable treatment products and individual treatment tips and/or (b) determine and/or display the treatment success of a cosmetic treatment that aims to positively influence the measured parameters.

The registration unit can have an interface (also: data transmission connection) via which a connection to the computing unit is established. The computer unit can have a first interface and a second interface. The first interface can be designed as a counterpart to the interface of the acquisition unit, i.e. to connect the acquisition unit to the computing unit. The second interface can be designed to connect the computing unit to a data network. These connections are designed to transmit information in at least one direction, preferably in both directions. The connection between the recording unit and the computing unit on the one hand and the connection between the computing unit and an access point of the data network can be wired or wireless. Wired connections can, for example, use optical or electrical signals to transmit information. Wireless connections typically use electromagnetic waves for signal transmission, e.g. radio signals or even optical signals.

Protocols that work according to the principles of mesh networks can be used to connect the acquisition unit to the computer unit. For example, the thread protocol, which is based on IPv6, can be used for data transmission and for connecting the registration unit to the computing unit. The thread protocol is used to connect automated or semi-automated devices with each other, in this case for example the acquisition unit with the computing unit.

In an example, the acquisition unit can be structurally attached to the computing unit, or vice versa. This means that the detection unit is mechanically attached to the computing unit or a housing of the computing unit. For example, this can be achieved by tool-free installation via a reversible connection. In the attached position, the registration unit can be held relative to the calculation unit by employing a detachable force-locking or form-fit connection. The interfaces between the registration unit and the computing unit can be arranged in such a way that an electrical connection between the registration unit and the computing unit is automatically established or established in the plugged-on position.

The computing unit can execute an application (or program, hereinafter also referred to as computer program product) that receives or queries data from the acquisition unit. The data received or queried is used in the application to determine one or more output values. The data is processed and/or evaluated by the application according to the approaches described herein.

To run the application, processors (and one or more memory modules) of the computing unit can be used. However, the calculation unit can also be designed to outsource calculation steps for the execution of the application. For example, the application can transfer the data received from or requested by the acquisition unit to an external processing unit. Before the data is transferred to the external computing unit, it can be pre-processed.

The external computing unit can be located at a distance from the acquisition unit and the portable computing unit. The portable computing unit can be connected to the external computing unit via the data network, i.e. be in a communication link. The external computing unit can be a single computer or processor or a network of computers or processors. In a computer or processor network, the computing load can be distributed to the individual components of the network under different aspects. In addition to computing power, this computer network can also provide storage capacity for the users and hold data released or marked by the users. This reduces the amount of memory required in the portable computing unit. It is also made easier for the user to exchange a portable computing unit because no or almost no data is stored locally. The computer network can be designed as a group of intermeshed networked servers.

According to another version, the evaluation unit is designed to compare the characteristics of treatment products for the treatment of hair with the determined hair properties and to determine an effect of the treatment products on the hair taking into account the determined hair properties.

This means that the treatment products are selected and determined according to the hair properties determined. These treatments can be displayed on the user interface, for example, or output in other ways.

According to another version, the evaluation unit is designed to transfer the determined hair properties to a data storage unit and to request information on the treatment of the hair according to the determined hair properties from the data storage unit.

These instructions can be general instructions (without reference to a specific treatment product) concerning the treatment and/or care of hair, but they can also be instructions with reference to a specific treatment product. The instructions may also include explanations of which behaviors influence which properties of the hair and how.

The data storage unit may contain information from studies and information from literature sources and/or scientific publications. The evaluation unit can be designed to provide a user with an extract of this information, or at least to draw attention to it, depending on the recorded characteristics of the hair.

According to another version, the arrangement still has a user interface, and the evaluation unit is designed to instruct the user interface to output the instructions received for treating the hair.

The user interface can, for example, be a display of the portable computing unit, in particular a so-called touch screen, which enables contents to be displayed visually and user input to be received by touch. Information can be output alternatively or additionally also auditively.

According to another version, the evaluation unit is designed to request information from a user and to take this information additionally into account when requesting the data storage unit in order to obtain from the data storage unit characteristics of treatment products for the treatment of hair according to the information requested by the user.

The information requested can be collected by employing a predefined questionnaire, in which a statement by the user is given weight or is selected from one of several possible answers. The prescribed questionnaire can deal in particular with the user's habits and extraordinary stresses and strains, e.g. dietary habits, duration and quality of sleep, amount drunk, type of drinks, use of stimulants (e.g. nicotine, alcohol), professional and leisure activities (spending a lot of time outside buildings in all weathers, staying in the mountains, visiting a solarium). The age, gender and ethnicity of the users can also be queried and used to query the treatment product data store. The requested information can also refer to a desired or achievable property of the hair.

According to another version, the evaluation unit has a local memory, which is designed to store the data retrieved by the data storage unit, preferably persistently.

This means that the evaluation unit can perform its functions at least temporarily without having to rely on a permanent connection to the data storage unit but the data network. The retrieved data is stored in the local memory. The data are stored in the local memory in such a way that they are retained when the evaluation unit is switched off or put out of operation (persistent storage). It is possible that the evaluation unit will only call up data from the data storage unit that matches a current image or current properties of the hair. It is also possible to retrieve and locally save data that matches slightly changed properties of the hair based on the current state. It is therefore not necessary to retrieve all data from the data storage unit and store it in the local memory. Rather, it is possible to transfer specific data or information from the data storage unit to the local memory that matches the recorded condition of the hair.

According to another version, the evaluation unit is designed to store the determined hair properties in the local memory with a time stamp concerning the determination of the hair properties.

This makes it possible to observe and analyze changes in the hair over time. Thus, these changes can also be used to issue appropriate non-therapeutic treatment products and/or treatment instructions. It also allows the user to monitor the changes to determine the achievement or approximation of self-defined goals.

According to a further version, the evaluation unit is designed to store the determined hair properties over a longer period of time comprising at least two processes of recording the hair properties in the local memory and optionally to call up a development of the hair properties over a specifiable period of time from the local memory and to instruct the output unit to output this development.

According to another version, the evaluation unit is designed to transfer the determined hair properties to the data storage unit.

The determined and transmitted hair properties can be assigned to a code number or a user ID in the data storage unit, so that a user can view his data from different devices. This procedure also has the advantage that a user's data is saved or stored at a central location in the event of loss or defect of the personal evaluation unit.

Furthermore, this design form makes it possible to record a user's hair characteristics over a longer period of time and to observe their changes and, if necessary, link them to the recommendations for non-therapeutic treatment agents and/or treatment instructions.

According to another version, the arrangement is designed to issue instructions for operating the registration unit visually on the output unit and/or acoustically via a loudspeaker. This can be particularly helpful when a user's hair is being recorded for the first time and comprehensively to gain an overview of the condition of the hair.

According to another version, the output unit is designed to output information about a treatment agent, e.g. a product name, information about ingredients and/or composition of a treatment agent and/or application instructions for non-therapeutic treatment of the hair.

This enables a user to form his or her own opinion about a treatment product in its entirety. In addition, the user can be given instructions for use related to a treatment product or independently of it. The application instructions can refer to desired and/or undesired behavior.

According to another version, the user interface is designed to receive an input from a user after the output of characteristics of a treatment agent and to initiate an action concerning the displayed treatment agent based on this input.

The action may, for example, relate to the user being offered a treatment product for sale and the user being able to initiate the purchase by employing an input. In addition to the purchase of treatment products, the user can also be offered more detailed information on the purchase. This more detailed information may refer to more detailed treatment and application instructions. For example, the program receives the request that the user wishes to purchase the treatment product, stores the request and/or transmits the request to a commercial company that distributes the treatment products. The user is requested by the computer program to enter his personal data (address, bank information, shipping preference, etc.) via the input unit.

Alternatively, the user can be dispensed where, for example in a drugstore, hairdressing salon, pharmacy, etc. in his vicinity, he can purchase the dispensed treatment product locally.

More and more customers want a product individually tailored to their needs. Accordingly, the user can be recommended an individually manufactured treatment product and an order process can be initiated, for example by calling up the website of a manufacturer of individual hair treatment products.

This may be a treatment product specially manufactured for one user or a so-called “mass customized” product. In the case of a “mass customized” product, individualization can be achieved by varying a few features of a product that are decisive from the customer's point of view. These “mass customized” products are preferably based on the concept of modularization, i.e. the product can be individually assembled from various modules/components.

There are often numerous interdependencies between the many different characteristics/ingredients of a product, which can be expressed as “commandments” or “prohibitions”. To obtain a clear product definition, it can be advantageous to use a product configurator during the ordering process. This configurator helps the user to select the features/ingredients and draws his attention to the allowable/non-permissible combinations of features, the latter of which cannot then be selected.

In the case of hair treatment products, the relevant product characteristics include the chemical ingredients of the products, the physical properties of the products and the way in which the products are packaged. With the help of a product configurator, for example, the selection of chemically and/or physically incompatible ingredients or the selection for the determined degree of damage/strain/etc. of unsuitable ingredients can be avoided. Conversely, the selection for the determined degree of damage/strain/etc. of suitable ingredients can be specified or suggested by the product configurator.

It is also possible to produce an individual hair treatment product on site, i.e. for example in a hairdressing salon or at a point of sale of hair treatment products, such as a drugstore, using a mixing device, preferably a smart mixer.

According to another aspect, a method to determine a reductive damage of hair is given. The procedure has the following steps: Irradiating a hair sample with electromagnetic waves in the near infrared region at a wavelength between about 800 and about 2500 nm; detecting light emitted from the hair sample; generating a spectrum of the emitted light; determining the reductive damage of hair based on the spectrum of the emitted light in a wavelength range between about 1970 and about 1980 nm.

This procedure corresponds to the function of the arrangement described above. The process steps correspond to the functions of the arrangement and are not described again here. In any case, reference is made to the above description of the order for details of the procedural steps. The other functions of the arrangement can of course also be implemented as process steps.

According to an implementation form, the procedure has the following step: Match the spectrum of the emitted light with known and available spectra of hair samples and assign a damage category to the hair sample based on this match.

According to another aspect, a method for determining a treatment agent based on the specific reductive damage of hair is given. This procedure has the following steps: Using the determined reductive damage of hair and selecting a treatment agent for hair based on the determined reductive damage and outputting information about the selected treatment agent.

According to one version, the procedure for determining a treatment agent has the following step: Selecting the treatment agent based on a desired treatment result.

The treatment product is selected or determined depending on the determined or specific reductive damage and considering a desired effect, for example desired properties of the hair after the treatment. The desired effect can be a user-defined effect or a desired condition of the hair. It can be helpful here to assign to each treatment agent one or more types of damage for which an application is possible and an effect which the treatment agent has on the corresponding type of damage. Thus, a simple comparison of the type of damage and the desired effect can be used to determine the appropriate treatment agent.

According to another aspect, a computer program product is specified which is designed to perform the procedure as described herein when executed on a device as also described herein.

The computer program product allows the control and follow-up of the results by displaying (e.g. graphically) the measurement results over time. Based on the results obtained, the computer program product provides individual treatment and product tips. The quality of the treatment and product tips can be improved by the user answering additional questions about his hair condition, dietary habits, general health, and other behavior that the computer program product can process accordingly. This is based not only on e.g. literature data, but also on the treatment success of other users of the computer program product, especially treatment successes of other users who have at least a similar hair condition.

The data collected through the questionnaire can be used to analyze a development of the condition of the user's hair under the given circumstances, i.e. the data entered by the user. This development can be compared with the development of other users. From this, it can be concluded whether, during treatment with a given product, the evolution of users with similar or identical submissions in the questionnaire is similar or different from users with different submissions.

For example, it is possible to draw conclusions about the influence of a certain fact on the success of the treatment. If, for example, the development of one type of damage in several smokers with a certain cigarette consumption (e.g. about 10 cigarettes per day) shows a significant deviation from the development of the same type of damage in non-smokers, it may be concluded that smoking has an effect on the particular parameter in a way that can be quantified. Alternatively, it can be concluded that a different product or treatment is recommended for smokers.

The data entered by the user can thus be used for a global analysis in order to monitor the success of a treatment and the effectiveness of a product under different conditions and, if necessary, recommend changes to the treatment and/or product.

In the following description, reference is made to the attached drawings which form part of the present application and which, for illustrative purposes, show specific embodiments in which the present disclosure can be exercised. It is understood that other embodiments may be used and structural or functional or logical modifications may be made without departing from the scope of protection of the present disclosure. In this respect, directional terminology such as “top”, “bottom”, “front”, “back”, “front”, “rear”, etc. is used with reference to the orientation of the figure(s) described. Since components of embodiments can be positioned in several different orientations, the terminology of directions is for illustrative purposes only and is in no way restrictive. It is understood that the characteristics of the various exemplary designs described herein may be combined, unless specifically stated otherwise. The following detailed description is therefore not to be understood in a restrictive sense and the scope of protection of the present disclosure is defined by the appended claims and equivalents thereof.

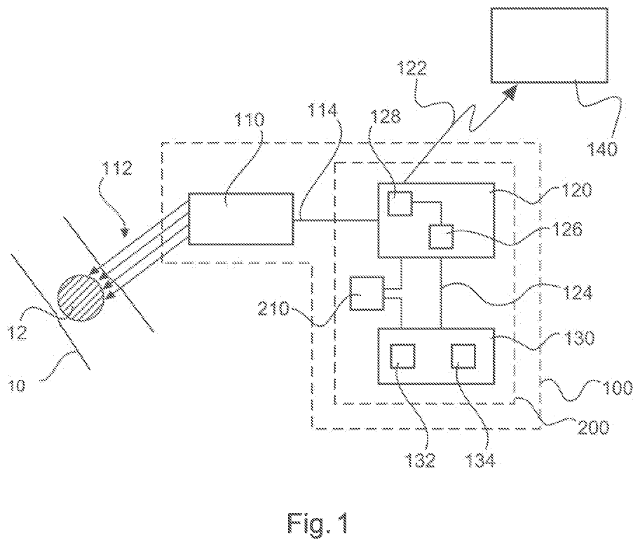

FIG. 1 shows an arrangement 100 to determine a reductive damage of hair. Arrangement 100 has an acquisition unit 110, an evaluation unit 120 and a user interface 130. The detection unit 110 is designed to detect properties of hair. For this purpose, the detection unit emits 110 light in a wavelength range between about 800 nm and about 2500 nm towards a region 12 of the analyzed object 10 (e.g. human hair) to be examined and detects emitted light especially in the wavelength range between about 1970 and about 1980 nm (including these two values). The light emitted from the area 12 to be examined is picked up by the detection unit 110 and allows conclusions to be drawn about the reductive damage to the hair, because the thiols that occur in the event of reductive damage to the hair have a harmonic in the wavelength range detected.

The detection unit 110 has a suitable source of electromagnetic waves. This source is a light emitter or laser emitter, also known as a radiation source, and is located on or in the detection unit 110. The radiation source may be placed on or in the detection unit 110 in such a way that when the electromagnetic waves 112 are emitted, the radiation source occupies a predetermined distance from the area 12 to be examined, in particular when the detection unit 110 is placed on the area to be examined. The distance of the radiation source from the area to be examined can be variable and can be changed by actuators or manually.

The registration unit 110 is connected to the evaluation unit 120 via a data transmission connection 114. The data transmission connection 114 can enable unidirectional or bidirectional data transmission between the acquisition unit 110 and the evaluation unit 120. Thus, the detection unit 110 delivers signals concerning the detected characteristics of the hair to the evaluation unit 120, whereas the evaluation unit 120 can deliver control commands to the detection unit 110, whereby the control commands determine how the detection unit 110 operates. In the case of a unidirectional data transmission connection 114, which only allows data transmission from the acquisition unit 110 to the evaluation unit 120, control parameters can be specified via input elements (buttons, switches, rotary knobs, etc., not shown) on the acquisition unit 110. The registration unit 110 may have display elements (not shown) that indicate a status of the registration unit or the set control parameters. Alternatively, the registration unit 110 can also transmit the set control parameters to the evaluation unit 120, where they can be optionally displayed.

The evaluation unit 120 has a processor 126 and a local memory 128. The evaluation unit 120 receives signals concerning the characteristics of the examined area 12 and determines a recommendation for a non-therapeutic treatment of the examined hair based on these characteristics. The non-therapeutic treatment may include recommendations on treatment products and/or treatment instructions or application instructions for the hair examined Treatment and application instructions are used as synonyms in the context of this description and refer to instructions for non-therapeutic treatment of the examined area (hair) 12 using selected treatment products or even without the use of treatment products. Treatment instructions may include the use of a treatment agent, or measures to be taken or not to be taken by the user. For example, the treatment instructions may include an indication of desirable or undesirable behavior after the use of a treatment product. To determine a non-therapeutic treatment to be recommended, the recorded characteristics of the investigated area 12 can be compared with areas of application, effects, and instructions for use of treatment agents and/or treatment instructions. Information on the treatment agents and/or treatment instructions can be stored in a data storage unit 140.

The data storage unit 140 can be located outside and spatially separated from the evaluation unit 120. The evaluation unit 120 can access the data storage unit 140 via a data network 122 and call up information on the treatment products stored there and/or treatment instructions. This retrieved information is compared by the evaluation unit 120 with the recorded characteristics of the examined area 12 to determine appropriate recommendations for the non-therapeutic treatment of the examined hair. In other words, this means that the data storage unit is queried using the determined hair properties. From the data storage unit, a large amount of stored information can first be retrieved and then filtered using the hair properties determined and, if necessary, treatment targets to determine which of the treatment agents and/or treatment instructions are relevant. For this purpose, the data can be loaded from the data memory into a volatile working memory. Alternatively, the determined hair properties can already be used when retrieving the information from the data memory to retrieve only the relevant information from the data memory. For the purposes of this description, these two variants can be considered equivalent in their effect.

The data network 122 may be a public data transmission network comprising sections of wire or wireless transmission. For example, the evaluation unit 120 may establish a wireless connection to an access point (not shown) to the data network 122 to establish a corresponding connection to the data storage unit 140.

The user interface 130 is connected to the evaluation unit 120 via the data transmission connection 124. The user interface 130 has an input unit 132 and an output unit 134. The input unit 132 enables a user to set parameters for the operation and configuration of the evaluation unit 120, the registration unit 110 and/or the user interface 130. Input unit 132 can record information via various interfaces: a keyboard, a mouse, a touch-sensitive display or via a microphone (so-called voice control). It is conceivable that any interface is used via which a human user can communicate with a computing unit and enter or transfer data. The output unit 134 can be a display or other display unit that provides visual information to a user. The output unit 134 can also have a loudspeaker via which acoustic information can be output. Visual information can be output on a touch-sensitive output unit so that the output unit also allows a user to make entries.

The evaluation unit 120 has a processor 126 and a local memory 128. The processor 126 executes instructions to perform its intended function or functions. The local memory 128 can store the characteristics of the hair detected by the detection unit 110 or the associated signals or values.

It is a special aspect of this design example that the registration unit 110 can be operated with an evaluation unit 120 and a user interface 130, which are implemented in a portable device of a user or consumer. This makes it particularly easy to couple a registration unit 110, which provides advanced analysis and examination possibilities for the hair of a human user, with a portable computerized data processing device. The portable data processing device can be, for example, a smartphone or tablet and a home computer. The registration unit 110 can be mechanically, electrically, and signal-wise connected or coupled to the portable data processing device via a defined interface.

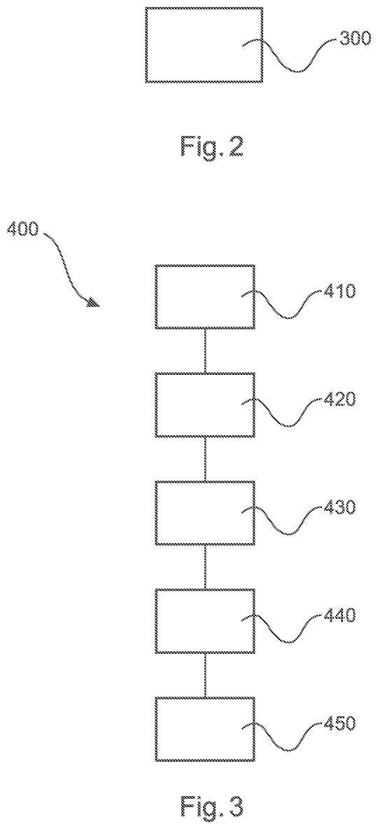

FIG. 2 shows a data carrier 300. A computer program product is stored on the data medium, which is designed to be executed on a portable computing unit 120 and to instruct a processor 126 of the portable computing unit to perform the following steps (cf. also the process steps in FIG. 3) Irradiating (410) a hair sample with near infrared electromagnetic waves at a wavelength between about 800 and about 2500 nm; detecting (420) light emitted from the hair sample; generating (430) a spectrum of the emitted light; determining (440) the reductive damage of hair based on the spectrum of the emitted light in a wavelength range between about 1970 and about 1980 nm.

The 300 medium may use magnetic, optical, or electrical storage techniques (or combinations thereof) to hold the instructions of the computer program product in a machine-readable form. These instructions can be used to be executed directly by the processor 126 of a portable calculation unit 120 (the evaluation unit 120 from the execution example in FIG. 1). Alternatively, the instructions can be used to load the computer program product into an internal memory of the portable computing unit 120 for execution. This internal memory can be the local memory 128 shown in FIG. 1.

The data carrier 300 can be a mobile and/or portable data storage device. Alternatively, the computer program product can also be loaded via a data network by accessing the data carrier 300 from a portable computing unit via the data network to load the computer program product via the data network. The computer program product can be downloaded via a data network to a user's portable device and installed on the portable device for use by the user.

In addition to FIG. 2, FIG. 3 shows a procedure 400 with the following steps (these steps correspond to the functions of the computer program product): Irradiating (410) a hair sample with near infrared electromagnetic waves at a wavelength between about 800 and about 2500 nm; detecting (420) light emitted from the hair sample; generating (430) a spectrum of the emitted light; determining (440) the reductive damage of hair based on the spectrum of the emitted light in a wavelength range between about 1970 and about 1980 nm; and matching (450) the spectrum of the emitted light with known and available spectra of hair samples and assigning a damage category to the hair sample based on this matching.

The computer program product contains instructions that instruct the processor 126 of the portable calculator 120 to perform these steps 410 to 450.

Of course, the procedure 400 or its steps 410 to 450 can be modified in accordance with one of the execution examples of arrangement 100, as shown with reference to FIG. 1 and the other description. This means that the functions of arrangement 100 or one of its components described herein, the evaluation unit 120, can be implemented as step of procedure 400. It is not necessary to repeat the functions of the evaluation unit at this point. Rather, the expert will recognize that and how these functions are implemented as procedural steps.

The different process steps as well as the components of the arrangement can be realized by one or more circuits. In an embodiment, a “circuit” is to be understood as any entity that implements a logic, which may be hardware, software, firmware, or a combination thereof. Thus, a “circuit” in one embodiment may be a hard-wired logic circuit or a programmable logic circuit, such as a programmable processor, e.g. a microprocessor or a field programmable gate array (FPGA) device. A “circuit” can also be a processor that executes software, e.g. any kind of computer program, such as a computer program in programming code for a virtual machine (delimited runtime environment, virtual machine), such as a Java computer program. A “circuit” can be understood as any type of implementation of the functions described below.

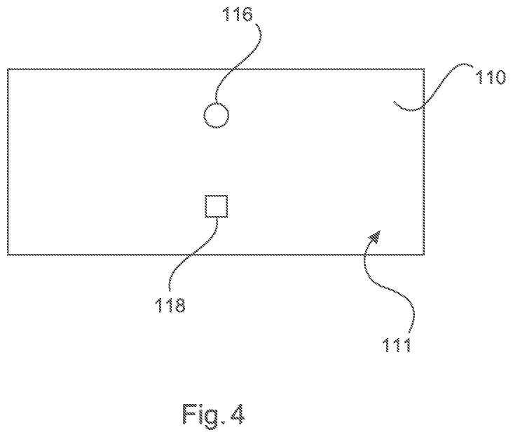

FIG. 4 shows a schematic diagram of a registration unit 110. The detection unit has a surface 111 on which a light emitter 116 and a spectrometer 118 are shown. The light emitter 116 is shown circular and the spectrometer 118 is shown square.

The surface visible from the detection unit 110 is that which faces the user's hair during a detection process. In other words, the light emitter 116 emits the light rays from the drawing plane towards an observer.

When the hair of a human user is irradiated with light (e.g. laser), part of this light is emitted depending on the chemical composition of the hair.

The processor 126 (FIG. 1) can implement control functions and issue control commands to the light emitter 116. For example, the processor 126 can control the light emitter to emit light of a certain intensity, wavelength and/or spectral distribution (these can be called parameters of light).

The evaluation unit 120 with the processor 126 (FIG. 1) also receives the signals from the spectrometer 118 and can classify the examined hair based on these signals. In other words, the signals delivered by the 118 spectrometer are characteristic of the examined hair. These signals can also be called signal patterns and can be used to determine and output a product recommendation and/or application notes.

It is conceivable that a typical signal pattern is assigned to a product and/or an application note, where the product and/or the application note can be sensibly applied to the examined hair to achieve a desired treatment result. This assigned signal pattern of the products and/or application notes can be compared with the actual signal pattern from the detection unit. From a certain degree of conformity of the signal pattern detected or supplied by the spectrometer with the signal pattern assigned to the products and/or application notes, the corresponding products and/or application notes can then be issued. The signals can be examined for qualitative similarity (do the shapes or courses of the signals correspond) and/or quantitative similarity (do the signals have similar input values, i.e. light, similar output values, i.e. emitted light).

It is also conceivable that, depending on user input, a factor may be determined and applied to the signal detected by the spectrometer before this input signal is compared with the signal patterns of the products or application notes. This has the advantage that a correction factor can be applied to the acquired signal to improve the accuracy of product recommendations and/or application notes for a particular user.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. An arrangement for determining a reductive damage of hair, comprising:
a detection unit for detecting hair characteristics thereby generating detected hair characteristics; and
an evaluation unit for evaluating the detected hair characteristics and for determining the reductive damage of hair based on the detected hair characteristics;
wherein the detection unit includes a near infrared sensor, NIRS;
wherein the detection unit is configured to irradiate a hair sample with electromagnetic waves in the near infrared region at a wavelength of from about 800 to about 2500 nm and to detect light emitted from the hair sample;
wherein the detection unit is configured to generate a spectrum of the emitted light and to provide the spectrum to the evaluation unit; and
wherein the evaluation unit is configured to determine a reductive damage based on the spectrum of the emitted light in a wavelength range of from about 1970 to about 1980 nm.

2. The arrangement according to claim 1,
wherein the detection unit is configured to emit the light emitted in a direction of the hair sample to be examined over an entire specified wavelength range of from about 800 to about 2500 nm.

3. The arrangement according to claim 1,
wherein the evaluation unit is configured to set an intensity of the emitted light in the wavelength range of from about 1970 to about 1980 nm in relation to the wavelength ranges below and above from the wavelength range of the emitted light to determine the reductive damage based on a pattern of the spectrum of the emitted light.

4. The arrangement according to claim 1,
wherein the evaluation unit is configured to compare the spectrum of the light emitted by the hair sample with known and available spectra of hair samples and to assign a damage category to the hair sample based on the comparison.

5. The arrangement according to claim 1,
wherein the near infrared sensor is a spectrometer, the spectrometer being configured to generate a signal pattern characteristic of the hair properties based on the detected light.

6. The arrangement according to claim 1,
further comprising a housing and an energy storage device, wherein the evaluation unit is accommodated in the housing and the detection unit is coupled to the housing, and wherein the energy storage device is arranged in the housing to supply the evaluation unit with energy and to enable at least temporarily a self-sufficient operation of the evaluation unit without connection to an external energy source.

7. The arrangement according to claim 1,
wherein the evaluation unit is designed to compare the features of treatment agents for treating hair with the determined hair properties to determine an effect of the treatment agents on the hair including taking into account the determined hair properties.

8. The arrangement according to claim 1,
wherein the evaluation unit is configured to transmit the determined hair properties to a data storage unit and to query from the data storage unit indications for treatment of the hair according to the determined hair properties.

9. The arrangement according to claim 8,
further comprising a user interface,
wherein the evaluation unit is designed to instruct the user interface to output the hair treatment instructions received.

10. The arrangement according to claim 8,
wherein the evaluation unit is configured to request information from a user and to additionally take the information requested into account when requesting the data storage unit to obtain from the data storage unit characteristics of treatment agents for treating hair in accordance with the information requested by the user.

11. A method for determining a reductive damage of hair, comprising:
irradiating a hair sample with electromagnetic waves in the near infrared range at a wavelength of from about 800 to about 2500 nm;
detecting the light emitted by the hair sample;
generating a spectrum of the emitted light; and
determining the reductive damage of hair based on the spectrum of emitted light in a wavelength range between about 1970 and about 1980 nm.

12. The method according to claim 11, further comprising:
matching the spectrum of the emitted light with known and available spectra of hair samples and assigning a damage category to the hair sample based on the match.

13. A method for determining a treatment agent based on the reductive damage to hair determined in claim 11, comprising:
using the specific reductive damage of hair; and
select a treatment product for hair based on the determined reductive damage and output information about the selected treatment product.

14. The method according to claim 13, further comprising:
selecting the treatment agent based on a desired treatment result.

15. A computer program product configured to perform the process according to claim 11 when performed in an order according to claim 1.
